(19) United States
(12) Patent Application Publication
(10) Pub. No.: US 2016/0271352 A1
(43) Pub. Date: Sep. 22, 2016

(54) NASAL BREATHING APPARATUS

(71) Applicant: Mount Royal International, Inc., Naples, FL (US)

(72) Inventors: Nikos Chintakis, Naples, FL (US); Kenneth Bookman, Naples, FL (US)

(21) Appl. No.: 15/073,736

(22) Filed: Mar. 18, 2016

Related U.S. Application Data
(60) Provisional application No. 62/134,850, filed on Mar. 18, 2015.

(51) Int. Cl. A61M 16/06; A61F 5/08

(52) U.S. Cl. CPC A61M 16/0666; A61F 5/08; A61M 2205/0216; A61M 2207/00

(57) ABSTRACT

A nasal breathing apparatus includes a first portion along a first axis and a second portion along a second axis. Each of the first portion and the second portion includes a passage, a plug section, and a passage section. The passage of the first portion extends along the first axis from a first opening at a first end of the first portion to a second opening at a second end of the first portion. The plug section of the first portion is near the first end of the first portion and has a diameter greater than a diameter of the passage section of the first portion at the second end of the first portion. The passage of the second portion extends along the second axis from a first opening at a first end of the second portion to a second opening at a second end of the second portion. The plug section of the second portion is near the first end of the second portion and has a diameter greater than a diameter of the passage section of the second portion near the second end of the second portion. A bendable bridge is arranged between the first and second portions.

### CROSS-REFERENCE TO RELATED APPLICATIONS

This application claims priority to U.S. Provisional Application No. 62/134,850 filed on Mar. 18, 2015.

### BACKGROUND

This disclosure relates to breathing aides, and more specifically to a nasal breathing apparatus.

Certain medical conditions cause breathing problems for a patient. For example, a patient may have difficulty breathing due to sinus congestion or sinus blockage. In other instances, problems related to a nasal passage, a patient's physical characteristics, or other known health-related conditions result in sleep apnea or snoring for the patient.

In certain circumstances, a physician, physician assistant, or other medical professional treats a patient using various intrusive devices to address the patient's particular condition. Typically, the patient is unable to insert such a device without the aid of the physician, physician assistant, or other medical professional. Moreover, breathing devices can be difficult to use and uncomfortable for patients during insertion and use due to the design of the device and materials used.

### SUMMARY

An example nasal breathing apparatus includes a first portion along a first axis and a second portion along a second axis. Each of the first portion and the second portion includes a passage, a plug section, and a passage section. The passage of the first portion extends along the first axis from a first opening at a first end of the first portion to a second opening at a second end of the first portion. The plug section of the first portion is near the first end of the first portion and has a diameter greater than a diameter of the passage section of the first portion near the second end of the first portion. The passage of the second portion extends along the second axis from a first opening at a first end of the second portion to a second opening at a second end of the second portion. The plug section of the second portion is near the first end of the second portion and has a diameter greater than a diameter of the passage section of the second portion near the second end of the second portion. A bendable bridge is arranged between the first and second portions.

An example method of inserting a nasal breathing apparatus includes a nasal breathing apparatus that includes a first portion along a first axis and a second portion along a second axis, wherein each of the first portion and the second portion includes a passage, a plug section, and a passage section, wherein the passage of the first portion extends along the first axis from a first opening at a first end of the first portion to a second opening at a second end of the first portion and the plug section of the first portion is near the first end of the first portion and has a diameter greater than a diameter of the passage section of the first portion near the second end of the first portion, wherein the passage of the second portion extends along the second axis from a first opening at a first end of the second portion to a second opening at a second end of the second portion and the plug section of the second portion is near the first end of the second portion and has a diameter greater than a diameter of the passage section of the second portion near the second end of the second portion, and a bendable bridge arranged between the first portion and the second portion. The first portion is inserted into the user's nasal passage through an opening at a first nostril such that the plug section of the first portion sit in proximity to the first nostril and dilates the opening at the first nostril. The second portion is inserted into the user's nasal passage through an opening at a second nostril such that the plug section of the second portion sit in proximity to the second nostril and dilates the opening at the second nostril. and the bendable bridge contacts the exterior of the user's nose

An example method of forming a nasal breathing apparatus includes forming a first portion along a first axis and a second portion along a second axis. Each of the first portion and the second portion includes a passage, a plug section, and a passage section. The passage of the first portion extends along the first axis from a first opening at a first end of the first portion to a second opening at a second end of the first portion and the plug section of the first portion is near the first end of the first portion and has a diameter greater than a diameter of the passage section of the first portion near the second end of the first portion. The passage of the second portion extends along the second axis from a first opening at a first end of the second portion to a second opening at a second end of the second portion and the plug section of the second portion is near the first end of the second portion and has a diameter greater than a diameter of the passage section of the second portion near the second end of the second portion. A bendable bridge is formed along a length between the first portion and the second portion.

### BRIEF DESCRIPTION OF THE DRAWINGS

FIG. 1A shows a perspective view of an example nasal breathing apparatus.

FIG. 1B shows another perspective view of an example nasal breathing apparatus.

FIG. 1C shows another perspective view of an example nasal breathing apparatus.

FIG. 1D shows another perspective view of an example nasal breathing apparatus.

FIG. 1E shows another perspective view of an example nasal breathing apparatus.

FIG. 2 shows a cross-sectional view of an example nasal breathing apparatus.

FIG. 3 shows an end view of an example nasal breathing apparatus.

FIG. 4 shows a cross-sectional view a user's nasal passage with an inserted example nasal breathing apparatus.

FIG. 5 shows a bridge of an inserted example nasal breathing apparatus.

### DETAILED DESCRIPTION

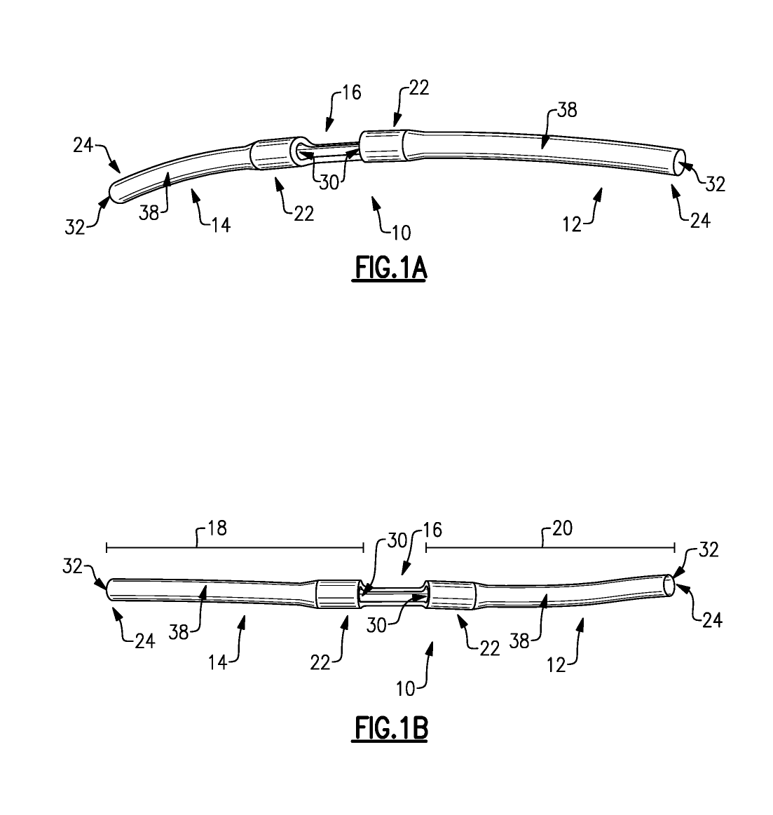

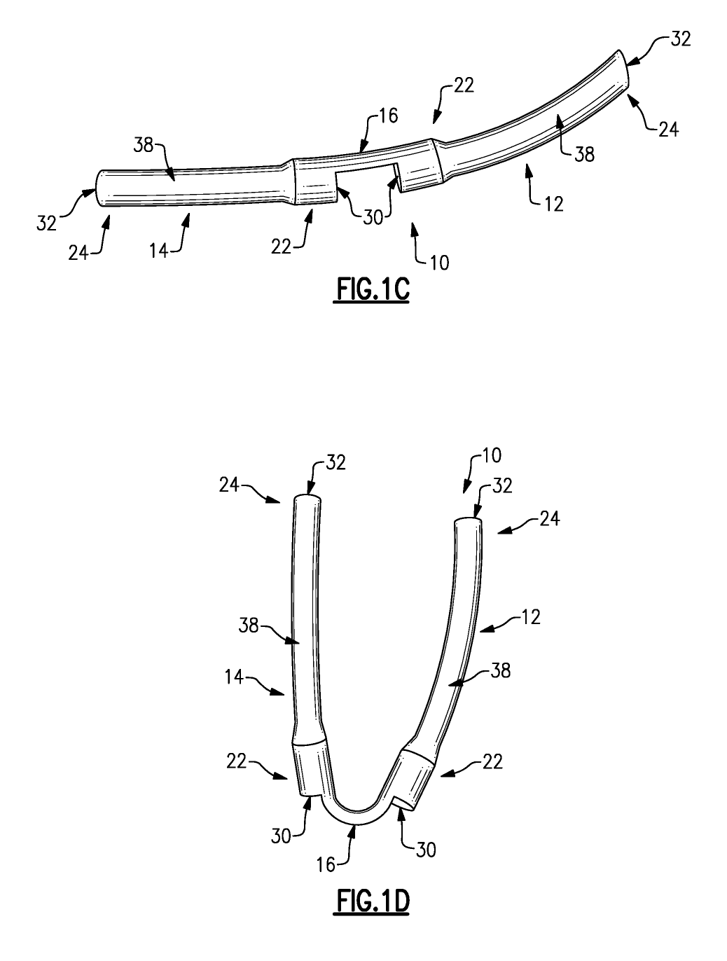

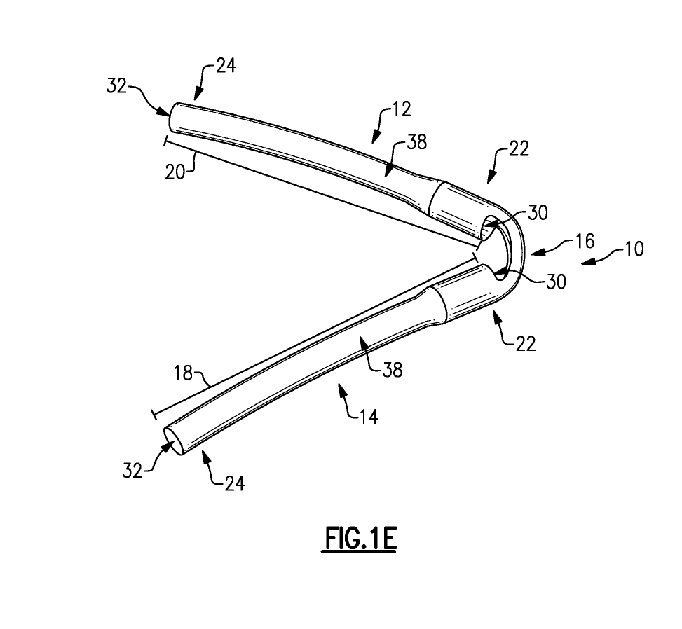

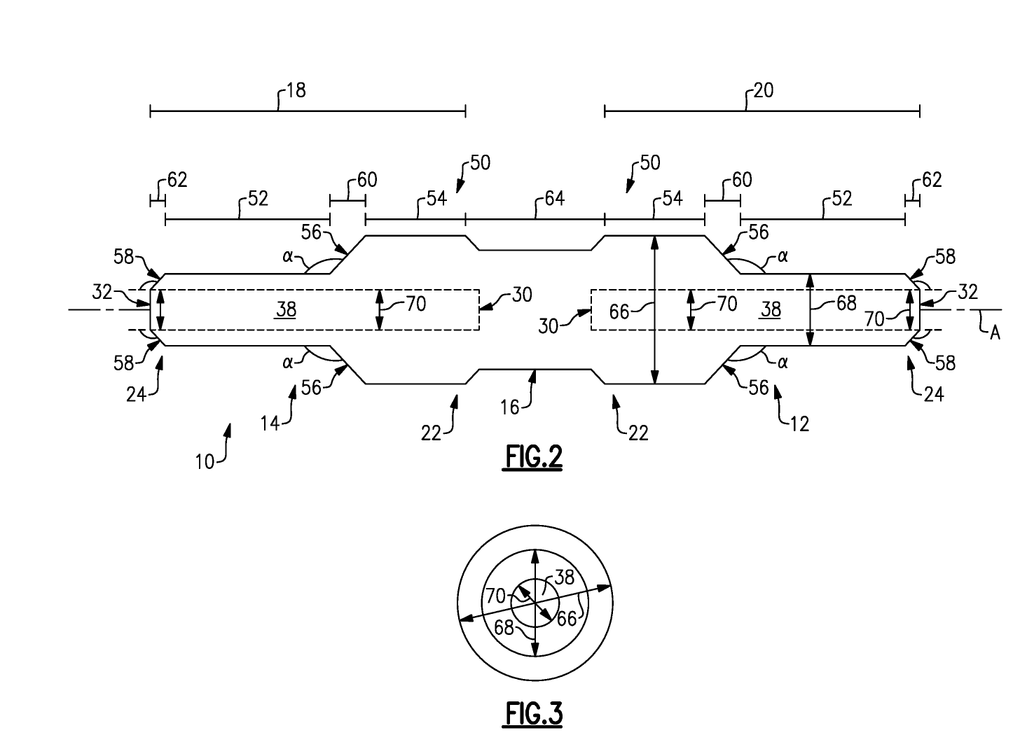

Referring to FIGS. 1A-1E, an example nasal breathing apparatus 10 includes a first portion 12 and a second portion 14 connected by a bridge 16. The first portion 12, the second portion 14, and the bridge 16 are integrally formed. The first portion 12 and second portion 14 each have a generally cylindrical profile with varying diameters, as will be described in further detail below. The generally cylindrical profile conforms to the shape of a nasal passage (as shown further in FIG. 5). However, other profiles may be used.

In this example, a length 18 of the first portion 12 and a length 20 of the second portion 14 are equal. However, in other examples, the length 18 and the length 20 may be different in response to a desired length corresponding to the nasal passage.

The first portion 12 and the second portion 14 each have a first end 22 near the bridge 16 and a second end 24 opposite the first end 22 and spaced away from the bridge 16. The bridge 16 extends between the first end 22 of the first portion and the first end 22 of the second portion 14 to connect the first portion 12 and the second portion 14. The bridge 16 is bendable to align the first portion 12 generally parallel to the second portion 14. The bridge 16 may be bent inwards or outwards such that the bridge 16 is disposed on the outer side of first ends 22, as shown in FIG. 1E, or on the inner side of first ends 22, as shown in FIG. 1D.

In one example, the nasal breathing apparatus 10 is made of a surgical grade plastic, or PVC. However, other materials may be used. In this example, the surgical grade plastic provides a soft, flexible material to allow the nasal breath apparatus 10 to be inserted by a user easily, as well as reducing discomfort during insertion and use. For example, the surgical grade plastic allows the nasal breathing apparatus 10 to conform to a slight curve of the nasal passage that may be present in certain users. The bridge 16 is bendable in any direction to allow adjustment of the first portion 12 and second portion 14 in the nasal passage.

Each of the first portion 12 and the second portion 14 includes a first opening 30 at the corresponding first end 22 and a second opening 32 at the corresponding second end 24. A passage 38 from the first opening 30 to the second opening 32 of each of the first portion 12 and the second portion 14 allows air to flow through each of the first portion 12 and the second portion 14. The air flows from the first opening 30 to the corresponding, connected second opening 32.

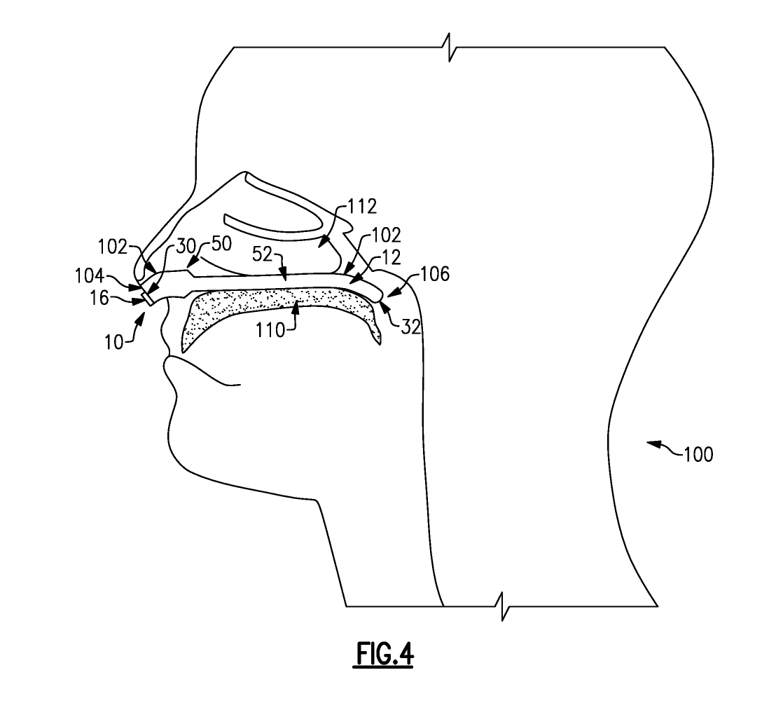

In one example, the nasal breathing apparatus 10 is manufactured by providing a cylindrical tube (not shown) of surgical grade plastic or other contemplated material, having a uniform thickness. The cylindrical tube is machined use of a grinder or buffer (not shown). In this manner, the thickness of the cylindrical tube is altered along differing lengths of the cylindrical tube to form the first portion 12 and second portion 14 with non-uniform diameters, as will be explained in further detail below. Additionally, tapers are formed between the varying diameters of each of the first portion 12 and the second portion 14 (as shown in FIGS. 2 and 3). Machining of the first portion 12 and the second portion 14 is done concurrently or consecutively. In another example, the nasal breathing apparatus 10 is manufactured by injection molding and includes any of the features described in this disclosure.

The cylindrical tube undergoes cutting, or other technique, to remove a section of a length and position corresponding to the length of the bridge 16. That is, a section of the cylindrical tube is removed to form the bridge 16, without severing the first portion 12 from the second portion 14. As a result, the integral nasal breathing apparatus 10 is formed. However, other methods of forming the nasal breathing apparatus 10 are contemplated, including but not limited to, permanently joining separate pieces of surgical grade plastic to form an integral nasal breathing apparatus 10 and using different plastic techniques to form the various features of the nasal breathing apparatus described herein.

Referring to FIGS. 2 and 3, with continued reference to FIGS. 1A-1E, each of the first portion 12 and the second portion 14 are disposed about an axis A and includes a plug section 50 and a passage section 52. Unless otherwise noted, description of the features shown in FIGS. 2 and 3 are provided with reference to the first portion 12 but are intended to also describe these same features in the second portion 14.

The plug section 50 begins at the first end 22 and extends a length 54 toward the corresponding second end 24. The passage section 52 is spaced from the plug section 50. The first portion 12 has a first taper 56 from the plug section 50 to the passage section 52. The first portion has a second taper 58 from the end of the passage section 52 opposite the plug section 50 to the second opening 32. In one example, the first taper 56 tapers from the plug section 50 to the passage section 52 at an angle α between 145° and 155° relative to the axis A. In one example, the second taper 58 tapers from the passage section 52 to the second opening 32 at an angle β between 40° and 50° relative to the axis A.

In one example, the first taper 56 extends a length 60 along axis A that is between 0.3 cm (0.118 inches) and 0.7 cm (0.276 inches). The second taper 58 extends a length 62 along axis A that is between 0.1 cm (0.039 inches) and 0.3 cm (0.118 inches). However, other taper lengths and angles α, β are contemplated by this disclosure. The first taper 56 and the second taper 58 provide a passage section 52 that is thinner, and of a lesser diameter, than the plug section 50 thereby making the nasal breathing apparatus 10 easier to insert into the nasal passage, more comfortable in the nasal passage, and less likely to irritate the user.

In one example, first portion 12 has a length 18 along axis A between 7 cm (2.76 inches) and 10 cm (3.94 inches), and the second portion 14 has a length 20 along axis A that is between 7 cm (2.76 inches) and 10 cm (3.94 inches). In one example, first portion 12 has a length 18 along axis A between 3.5 cm (1.38 inches) and 10 cm (3.94 inches), and the second portion 14 has a length 20 along axis A that is between 3.5 cm (1.38 inches) and 10 cm (3.94 inches). The bridge 16 has a length 64 along axis A that is between 1.5 cm (0.59 inches) and 3 cm (1.18 inches). The length 18 of the first portion 12, the length 20 of the second portion 14, and the length 64 of the bridge 16 are determined in response to a length of a nasal passage and bridging necessary between a left and right nasal passage. In one example, a ratio of the length 18 of the first portion 12 or the length 20 of the second portion 14 to the length 64 of the bridge 16 is between about 2.33 and 6.67. The lengths 18, 20, 64, are chosen to provide a nasal breathing apparatus 10 that reaches from the front of the nasal passage to the back of the nasal passage to provide airflow there through without causing discomfort by being inserted too far into the nasal passage and throat. However, other lengths are contemplated by this disclosure.

In one example, a ratio of the length 18 of the first portion 12 or the length 20 of the second portion 14 to the length 60 of the first taper 56 is between about 10 and 33.33. A ratio of the length 18 of the first portion 12 or the length 20 of the second portion 14 to the length 62 of the second taper 58 is between about 23.33 and 100.

In one example, the plug section 50 has a diameter 66 that is between 0.7 cm (0.276 inches) and 0.9 cm (0.354 inches). The passage section 52 has a diameter 68 that is between 0.5 cm (0.197 inches) and 0.7 cm (0.276 inches). The second opening 32 has a diameter 70 that is between 0.3 cm (0.118 inches) and 0.5 cm (0.197 inches). In one example, a ratio of the diameter 66 of the plug section 50 to the diameter 68 of the passage section 52 is between about 1.28 and 1.8. In one example, a ratio of the diameter 66 of the plug section 50 to the diameter 68 of the passage section 52 is about 1.4. A ratio of the diameter 70 of the second opening 32 to the diameter 68 of the passage section 52 is between about 0.43 and 1. However, other diameters of the first portion 12, second portion 14, second opening 32 and passage 38 are contemplated in this disclosure.

The diameter 70 of the second opening 32 is equal to the diameter 70 of the passage 38. The diameter 70 of the passage 38 is uniform from the second opening 32 to the first opening 30 of each of the first portion 12 and the second portion 14. In this manner, the first portion 12 and the second portion 14 utilize the plug sections 50, the passage section 52, and the second opening 32 having different diameters from different thicknesses at those locations. As a result, airflow through passage 38 is not restricted at any point along the first portion 12 or the second portion 14.

The larger diameter 66 of plug section 50 relative to the diameter 68 of the passage section 52 provides a nasal breathing apparatus 10 with a plug section 50 operable to sit near the front of the nasal passage, secure and fix the nasal breathing apparatus 10 in place, and dilate the nasal passage to increase airflow. In the same manner, the smaller diameter 68 of the passage section 52 and diameter 70 of the second opening 32 allow the nasal breathing apparatus 10 to pass through the nasal passage with minimal irritation to the user during insertion, and reduces discomfort during use. In one example, the diameter 70 of the passage 38 is sized such that airflow passes through the passage 38, but passage 38 is not sized or configured to receive additional tubes, instruments, or other medical devices. That is, in this example, nasal breathing apparatus 10 is freestanding and not in physical or fluid communication with any other device.

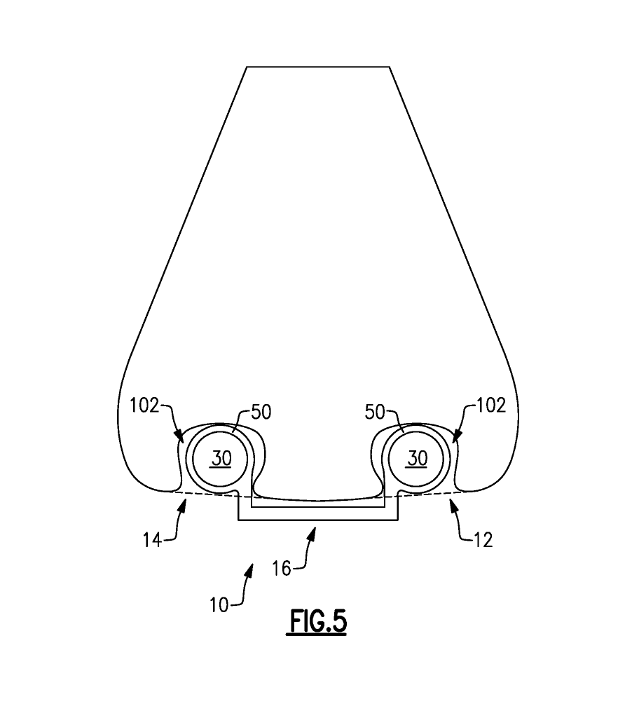

Referring to FIGS. 4 and 5, with continued reference to FIGS. 1A-1E, 2 and 3, the nasal breathing apparatus 10 is inserted by the user 100 into the user's 100 nasal passage 102. In one example, the user 100 is any patient, person, or individual who desires the nasal breathing apparatus 10 to aid in airflow through the nasal passage 102.

The nasal passage 102 has an inlet 104 at the beginning of the nostril of the user 100 and an outlet 106 at the upper aspect of the nasopharynx. The nasal breathing apparatus 10 is inserted beginning with the second opening 32 at the inlet 104 and passing through the nasal passage 102 until the second opening 32 is generally positioned near the outlet 106. When the second opening 32 is positioned near the outlet 106, the first opening 30 is positioned near the inlet 104, and passage 38 provides airflow from the inlet 104 to the outlet 106 of nasal passage 102. While in use, the first portion 12 and the second portion 14 are disposed between the hard palate 110, or floor, of the nasal passage 102 and the inferior turbinates 112 of the nasal passage 102.

In this example, the first portion 12 is inserted into the right nostril of the user 100 and the second portion 14 is inserted into the left nostril of the user 100. The first portion 12 and the second portion 14 are separated by the septum of the nose (not shown). The bridge 16 extends between the first portion 12 and the second portion 14 and contacts the nose outside of the nasal passage 102 to prevent the nasal breathing apparatus 10 from being inserted too far down the nasal passage 102. Plug section 50 sits in proximity to the inlet 104 of the nasal passage 102 and dilates the nasal passage 102. In one example, the first opening 30 is disposed outside of the nostril. In another example, the first opening 30 is disposed inside of the nostril.

In one example, when the bridge 16 is bent to align the first portion 12 and the second portion 14 generally parallel to each other, the passage 38 of each of the first portion 12 and the second portion 14 is curved. This curve provides easier access for the second opening 32 to the outlet 106 at the upper aspect of the nasopharynx. That is, the shape of the first portion 12 and the second portion 14 conform to the shape of the nasal passage 102.

The nasal breathing apparatus 10 of the present disclosure dilates the nasal passage 102 and provides increased airflow through the narrow nasal passage 102. The nasal breathing apparatus 10 is inserted by the user 100, reducing cost and expense associated with medical visits. The features of the nasal breathing apparatus 10 described herein further provide increased comfort and reduced irritation during insertion and use by the user 100.

It is understood that any of the herein described features may be used alone, or in combination in any nasal breathing apparatus 10. Although certain dimensions and parameters are described herein, other dimensions and parameters are contemplated and can be utilized. Dimensions and parameters described as related to example lengths, sections, or portions of the nasal breathing apparatus 10 are not limited to these lengths, sections, or portions and may be utilized with other lengths, sections, or portions.

Although certain herein described examples have a specific combinations of features shown in the illustrations, this disclosure are not limited to those particular combinations. It is contemplated to use some of the features from one of the examples in combination with features from another one of the examples.

Furthermore, the foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

1. A nasal breathing apparatus, comprising:
a first portion along a first axis and a second portion along a second axis, wherein each of the first portion and the second portion includes a passage, a plug section, and a passage section, wherein the passage of the first portion extends along the first axis from a first opening at a first end of the first portion to a second opening at a second end of the first portion and the plug section of the first portion is near the first end of the first portion and has a diameter greater than a diameter of the passage section of the first portion near the second end of the first portion,
wherein the passage of the second portion extends along the second axis from a first opening at a first end of the second portion to a second opening at a second end of the second portion and the plug section of the second portion is near the first end of the second portion and has a diameter greater than a diameter of the passage section of the second portion near the second end of the second portion; and
a bendable bridge arranged between the first portion and the second portion.

2. The nasal breathing apparatus of claim 1, wherein the first portion, the second portion, and the bendable bridge are integrally formed.

3. The nasal breathing apparatus of claim 2, wherein the first portion, the second portion, and the bendable bridge are made of surgical grade plastic.

4. The nasal breathing apparatus of claim 1, wherein the bendable bridge extends between the first opening of the first portion and the first opening of the second portion, wherein the bendable bridge is configured to align the first portion generally parallel to the second portion.

5. The nasal breathing apparatus of claim 4, wherein the diameter of the passage of the first portion is uniform and the diameter of the passage of the second portion is uniform.

6. The nasal breathing apparatus of claim 5, wherein the diameter of the passage of the first portion and the diameter of the passage of the second portion is between 0.3 (0.118 inches) and 0.5 cm (0.197 inches).

7. The nasal breathing apparatus of claim 4, wherein each of the first portion and the second portion have a length of between 7 cm (2.76 inches) and 10 cm (3.94 inches) and the bendable bridge has a length of between 1.5 cm (0.59 inches) and 3 cm (1.18 inches).

8. The nasal breathing apparatus of claim 7, wherein the diameter of the passage of the first portion and the diameter of the passage of the second portion is between 0.7 cm (0.276 inches) and 0.9 cm (0.354 inches), wherein the diameter of the passage section of each of the first portion and the second portion is between 0.5 cm (0.197 inches) and 0.7 cm (0.276 inches).

9. The nasal breathing apparatus of claim 4, wherein the first end of the passage section of the first portion is spaced from the plug of the first portion by a first taper and the first end of the passage section of the second portion is spaced from the plug of the second portion by a second taper.

10. The nasal breathing apparatus of claim 9, wherein an angle of the first taper relative to the first axis of the first portion and an angle of the second taper relative to the second axis of the second portion is between 145° and 155°, wherein each of the first taper and the second taper each comprise a length between 0.3 cm (0.118 inches) and 0.7 cm (0.276 inches).

11. The nasal breathing apparatus of claim 9, wherein the second end of the first portion includes a third taper extending to the second opening of the first portion and the second end of the second portion includes a fourth taper extending to the second opening of the of the second portion.

12. The nasal breathing apparatus of claim 11, wherein an angle of the third taper relative to the first axis of the first portion and an angle of the fourth taper relative to the second axis of the second portion is between 40° and 50°, wherein the third taper and the fourth taper each comprise a length between 0.1 cm (0.029 inches) and 0.3 cm (0.118 inches).

13. A method of inserting a nasal breathing apparatus comprising a first portion along a first axis and a second portion along a second axis, wherein each of the first portion and the second portion includes a passage, a plug section, and a passage section, wherein the passage of the first portion extends along the first axis from a first opening at a first end of the first portion to a second opening at a second end of the first portion and the plug section of the first portion is near the first end of the first portion and has a diameter greater than a diameter of the passage section of the first portion near the second end of the first portion, wherein the passage of the second portion extends along the second axis from a first opening at a first end of the second portion to a second opening at a second end of the second portion and the plug section of the second portion is near the first end of the second portion and has a diameter greater than a diameter of the passage section of the second portion near the second end of the second portion, and a bendable bridge arranged between the first portion and the second portion, the method comprising the steps of:
inserting the first portion into the user's nasal passage through an opening at a first nostril such that the plug section of the first portion sit in proximity to the first nostril and dilates the opening at the first nostril; and
inserting the second portion into the user's nasal passage through an opening at a second nostril such that the plug section of the second portion sit in proximity to the second nostril and dilates the opening at the second nostril.

14. The method of claim 13, wherein the step of inserting the first portion and inserting the second portion occur simultaneously.

15. The method of claim 13, further comprising the step of bending the bendable bridge to align the first portion and the second portion generally parallel to one another prior inserting the first portion.

16. The method of claim 13, wherein the first portion and the passage of the second portion have a generally cylindrical profile to conform to the user's nasal passage during the inserting step.

17. The method of claim 13, wherein second end of the first portion is situated adjacent an upper aspect of the user's nasopharynx after insertion of the first portion and the second end of the second portion is situated adjacent an upper aspect of the user's nasopharynx after inserting the second portion.

18. The method of claim 17, wherein the passage of each of the first portion and the second portion is curved.

19. A method of forming a nasal breathing apparatus, comprising the steps of:
Forming a first portion along a first axis and a second portion along a second axis, wherein each of the first portion and the second portion includes a passage, a plug section, and a passage section, wherein the passage of the first portion extends along the first axis from a first opening at a first end of the first portion to a second opening at a second end of the first portion and the plug section of the first portion is near the first end of the first portion and has a diameter greater than a diameter of a passage section of the first portion near the second end of the first portion, wherein the passage of the second portion extends along the second axis from a first opening at a first end of the second portion to a second opening at a second end of the second portion and the plug section of the second portion is near the first end of the second portion and has a diameter greater than a diameter of a passage section of the second portion near the second end of the second portion; and
forming a bendable bridge along a length between the first portion and the second portion.

20. The method of claim 19, further comprising the steps of forming a first taper between the plug section of the first portion and the passage section of the first portion;
forming a second taper between the plug section of the second portion and the passage section of the second portion;
forming a third taper at an end of the passage section of the first portion opposite the plug section of the first portion and adjacent the first opening; and
forming a fourth taper at an end of the passage section of the second portion opposite the plug section of the second portion and adjacent the second opening.
